(19) United States
(12) Plant Patent Application Publication
(10) Pub. No.: US 2005/0262599 P1
(43) Pub. Date: Nov. 24, 2005

(54) Miniature rose plant 'JENtwo'

(50) ROSA HYBRIDA; MINIATURE ROSE

(76) Inventor: Svend Jensen, Christiansfeld (DK)

Correspondence Address: Stefan J. Kirchanski;Reed Smith, LLP, Suite 700, 1901 Avenue of the Stars, Los Angeles, CA, 90067, US

(21) Appl. No.: 10/848,786

(22) Filed: May 19, 2004

(51) Int. Cl. A01H 5/00

(52) U.S. Cl. PLT/122

(57) ABSTRACT

A new miniature rose plant which has abundant, long lasting, dark-red colored flowers and attractive glossy foliage. The variety successfully propagates from softwood cuttings and is suitable for year round production in commercial glass houses as a flowering pot plant. This new and distinct variety has shown to be uniform and stable in the resulting generations from asexual propagation.

### BACKGROUND OF THE INVENTION

The present invention constitutes a new and distinct variety of miniature rose plant, which was developed by artificially pollinating an unnamed seedling(not patent in the US) with an unnamed seedling (not patent in the US). The two parents were crossed in the summer of 2001 and the resulting seed was sown in December 2001, in a controlled glasshouse environment. Out of these seedlings one seedling was selected, as the new variety and named ‘JENtwo’. The new rose may be distinguished from its seed parent, an unnamed seedling, by the following combination of characteristics:
1. The unnamed seedling has a breeding background in unnamed seedlings.
2. ‘JENtwo’ has small double flowers, while the unnamed seedling has medium single flowers.
3. ‘JENtwo’ has dark-red colored petals, while the unnamed seedling has orange petals.

The new variety may distinguished from its pollen parent, an unnamed seedling created by the same inventor, by the following combination of characteristics:
1. The unnamed seedling has a breeding background in unnamed seedlings.
2. ‘JENtwo’ has smaller flowers and foliage as compared to the unnamed seedling.
3. ‘JENtwo’ has dark-red colored petals, while the unnamed seedling has white petals.

### BRIEF SUMMARY OF THE INVENTION

Initial asexual reproduction of ‘JENtwo’ by cuttings was first done in Christiansfeld, Denmark. The reproduction was conducted in controlled greenhouse environments. Have here proven that the foregoing and all after characteristics and distinctions to come true to form and are established in succeeding propagations. ‘JENtwo’ is a low and compact miniature rose with medium vigor. The shelflife for the entire plant and the single flush, has been tested to be medium.

The objective of the hybridization of this rose variety for commercial greenhouse culture was to create a new and distinct variety with:
1. Uniform and abundant flowers with good keepability;
2. Attractive long lasting foliage and compact growth, and young foliage has striking red edges,
3. Year round flowering under glasshouse conditions;
4. Suitability for production from softwood cuttings in pots.;
5. Durable flowers and foliage which make the variety suitable for distribution in the floral industry.

This combination of qualities was not present in previously available commercial cultivars of this type and distinguish ‘JENtwo’ from all other varieties of which we are aware. The seeds from hybridization were planted in a controlled environment and evaluations were conducted on the resulting plants. ‘JENtwo’ was selected by, Svend Jensen, in his development program in Christiansfeld, Denmark.

### BRIEF DESCRIPTION OF THE DRAWINGS

The accompanying color illustrations show as true as is reasonably to obtain in color photographs of this type, the typical characteristics of the buds, flowers, leaves, stems of ‘JENtwo’. Specifically illustrated in:

### Photo Sheet # 1

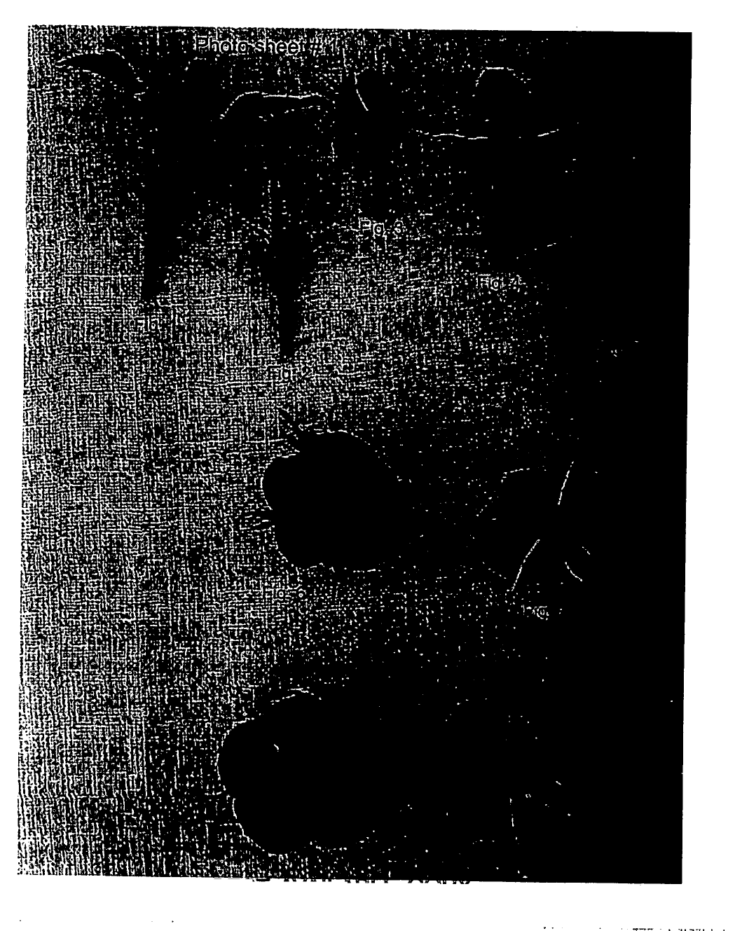

FIG. 1 Young shoot.

FIG. 2 Bud before opening the sepals.

FIG. 3 Bud at the stage opening the sepals.

FIG. 4 Bud at the stage opening the petals.

FIG. 5 Flower during course of opening.

FIG. 6 Open flower—plan view—obverse.

FIG. 7 Open flower—plan view—reverse.

FIG. 8 Fully open flower—plan view—obverse.

FIG. 9 Fully open flower—plan view—reverse.

### Photo Sheet # 2

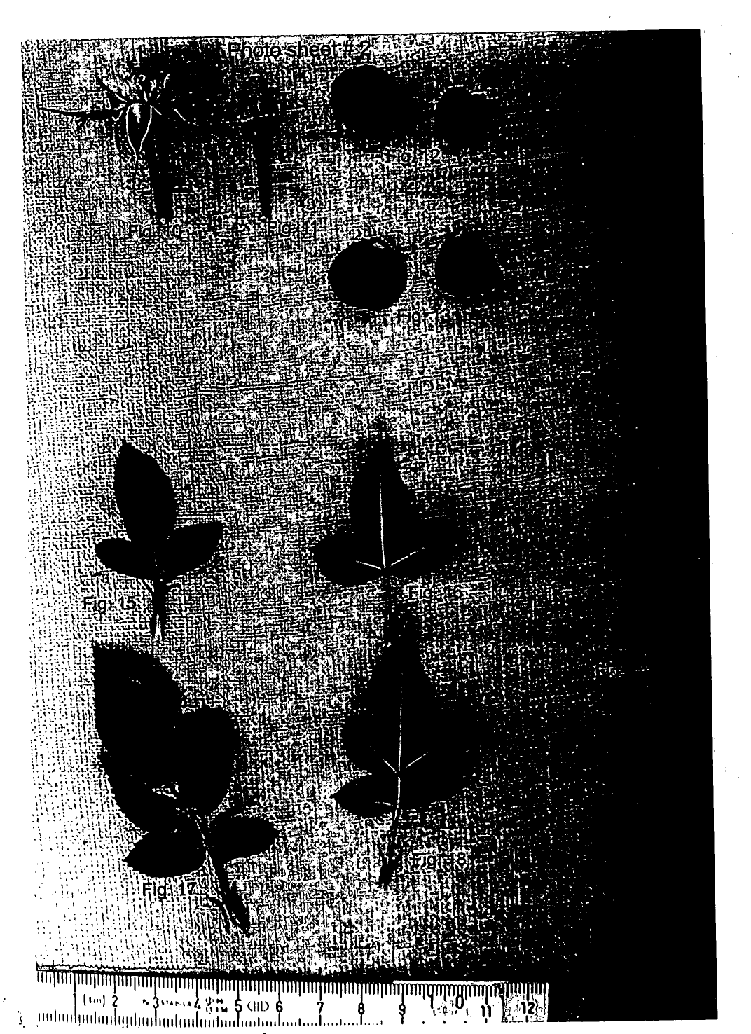

FIG. 10 Receptacle showing stamens and pistils.

FIG. 11 Receptacle showing pistils (stamens and sepals removed).

FIG. 12 Flower petals, detached—Inner surface.

FIG. 13 Flower petals, detached—Outer surface.

FIG. 14 Bare stem exhibiting thorns and flower attachment.

FIG. 15 Three leaflets upper side.

FIG. 16 Three leaflets reverse side.

FIG. 17 Five leaflets upper side.

FIG. 18 Five leaflets reverse side.

### DETAILED BOTANICAL DESCRIPTION OF THE VARIETY

The following is a detailed description of the Miniature Rose: Rosa hybrid ‘JENtwo’.

The following observations, measurements, values and comparisons describe plants grown in glass houses in Christiansfeld, Denmark. The age of the observed plants where 11 to 13 weeks after propagation by cuttings, and produced as a flowering pot plants in container of 10 centimeter in diameter. Color references are made using The Royal Horticultural Society (London, England) Colour Chart, 1995, except where common terms of color are used. For a comparison, the nearest existing rose variety is ‘Ruijef’, a rose variety described and illustrated in U.S. Plant Pat. No. 9,635.

Chart 1 details several physical characteristics of ‘JENtwo’ and ‘Ruijef’.

CHART 1

| | ‘JENtwo’ | ‘Ruijef’ |
|---|---|---|
| Petal color, Upper | Red Group | Red Group |
| surface | 46A | 46A |
| Petal color, Reverse | Red Group | Red Group 59 |
| surface | 46A | |
| Petal count | 20 | 35-45 |

Parents: Unnamed seedling. Times. Unnamed seedling
Classification:
   Botanical.—Rosa hybrid.
   Commercial.—Miniature.
Plant:
   Plant growth.—Moderately vigorous. Grows compact upright to bushy. When grown as 10 cm pot plant, the average height of the plant itself is 18 to 20 cm, and average width is 20 cm. When grown as a 15 cm pot plant, the average height of the plant itself is 22 to 27 cm, and average width is 30 cm. Production time is generally 11 to 13 weeks depending on average temperature, light level, and cultural practices.
Stem:
   Color.—Young wood: Yellow-Green Group 153 D. Older wood: Yellow-Green Group 152 B.
   Thorns.—Incidence: High number of thorns Size: 3-6 mm Color: Red Group 53 A Shape: Deep concave.
   Surface.—Young wood: Smooth Older wood: Smooth.
   Stem diameter.—3-4 mm.
   Internode length.—20-30 mm.
   Numbers of internodes.—6-8.
Plant foliage: Leaves arranged alternately, compound with 3, 5 or 7 leaflets per leaf, generally symmetrical, abundant, and flat in aspect. Stipules at petiole base.
   Quantity of leaves.—6-8 per lateral branch.
   Leaf size.—Medium 60-80 mm(l). times. 30-40mm(w), for 5 leaflet.
   Petioles.—Color: Yellow-Green Group 148A-C, Margins: with small prickles Red Group 53A. Length: 8-10 mm Diameter: about 1-2 mm.
   Stipules.—Size: 6-10 mm. Surface: Smooth Color: Yellow-Green Group 147A, With intonations of Greyed-Purple Group 185B Margins: serrated, single.
   Rachis.—Color: Yellow-Green Group 148A-C, With intonations of Greyed-Purple Group 185B Margins: With small prickles, Red Group 53 A. Length: 25 to 30 mm.
   Leaflets.—Edge: Serrated Serration: Double Shape: Ovate with acute apex and obtuse base Texture: Smooth Appearance: Glossy Size: length: 18 to 35 mm. With: 8 to 18 mm Color: Young foliage: Upper surface: Greyed-Red Group 181A Lower surface: Greyed-Red Group 180A, Color: Mature foliage: Upper surface: Green Group 147A Lower surface: Greyed-Green Group 191A With Intonations of Greyed-Red Group 181B.
Inflorescense:
   Blooming habit.—Recurrent
   Number of flowers.—Generally 1-3 bud per flowering stem.
   Peduncle.—Color: Yellow-Green Group 152B, Texture: Smooth, with stipitate glands Length: 20-25 mm Strength: Upright.
   Receptacle.—Surface: Smooth, glabrous. Shape: Cupped-shaped Size: h:5-6 mm w:6-8 mm Color: Yellow-Green Group 147B.
   Sepals.—Quantity: 5 Shape: Narrowly Ovate w. acute tip Texture: Leathery Margin: Foliaceous appendages on 3 of the five sepals Appearance: Dull Color: Upper surface: Yellow-Green Group 147A-B, Reverse surface: Greyed-Green Group 191A.
   Buds.—Size: 15-18 mm (h) 10-12 mm (w)upon opening. Shape: Cupped Color: at ¼ opening, Red Group 46 A.
Flower:
   Duration.—As a pot plant, flowers last from 18 to 21 days. Fragrance. None. Size: 40-45 mm in diameter. Form: Shape of flower when viewed from the side. Up on opening: Cupped Open flower: Cupped Color: Petals, upon opening. Upper surface: Red Group 46 A, Reverse surface: Red Group 46 A, Petals after opening: Upper surface: Red Group 46 A, Reverse surface: Red Group 46A. Basal Petals spots: Size: 1-2 mm Color: White Group 155 D. General tonality: On Open flower: Third day: Red Group 46A, Afterwards: Red Group 46B.
Petals:
   Petal reflex.—Outermost petals reflex backwards at opening . Fully open all petals reflex backwards.
   Texture.—Smooth.
   Petal edge.—Uniform.
   Petal count.—Approximately 20 on the average per flower.
   Petal size.—Length 13-15 mm Width: 10-20 mm.
   Shape.—Outer petals: Round. Inner petals: Ovate.
Reproductive organs:
   Stamen number.—Approximately 40 on average per flower.
   Pollen.—Color: Yellow-Orange Group 22D, Abundance: Average.
   Anthers.—Size: 1-2 mm Color: White Group 155D, Shape: Oblong.
   Filaments.—Size: 4- 5mm Color: Yellow-Orange Group 20D.
   Pistils number.—Approximately 20 on average per flower.
   Stigmas.—Location: Under in location to anthers Color: Green-White Group 157D.
   Styles.—Color: Red Group 53A. Length: 2to4 mm.
Development:
   Vegetation.—Dense.
   Blooming.—Abundant.
   Aptitude to bear fruit.—Poor.
   Resistance to diseases.—Above average resistance to mildew and Botrytis under normal growing conditions in Christiansfeld, Denmark. Hips/seeds has not been observed due to that the plant has never been grown to the stage of seed development, due to the fact, that the variety is developed for use as a flowering pot plant only.
   Winter hardiness & drought/heat tolerance.—Due to the fact, that this variety is a potted flowering plant, developed for one time use only (indoor houseplant), the plant is not tested for winter hardiness or drought/heat tolerance.

1. A new and distinct variety of rose plant of the miniature class, substantially as herein illustrated and described as a distinct and novel rose variety due to its abundant, long lasting, dark-red colored flowers, attractive long lasting glossy foliage, vigorous and compact growth, year round flowering under glasshouse conditions, suitability for production from softwood cuttings in pots, and durable flowers and foliage which make the variety suitable for distribution in the floral industry.
